(19) United States
(12) Patent Application Publication
(10) Pub. No.: US 2014/0134224 A1
(43) Pub. Date: May 15, 2014

(54) STABLE COMPOSITION OF HOCI, A PROCESS FOR ITS PRODUCTION AND USES THEREOF

(71) Applicants: Christopher Mallet, Wiltshire (GB); Christopher William Wederell, Rugby (GB); Richard Sinden, Worcestershire (GB)

(72) Inventors: Christopher Mallet, Wiltshire (GB); Christopher William Wederell, Rugby (GB); Richard Sinden, Worcestershire (GB)

(21) Appl. No.: 14/004,094

(22) PCT Filed: Mar. 8, 2012
(86) PCT No.: PCT/GB12/00228

§ 371 Date: Dec. 16, 2013

(30) Foreign Application Priority Data
Mar. 11, 2011 (GB) 1104180.3

(51) Int. Cl. A01N 59/00

(52) U.S. Cl. CPC A01N 59/00
USPC 424/408; 424/661; 424/409

(57) ABSTRACT

A stable antimicrobial aqueous hypochlorous acid solution that retains its activity for at least three months and can be provided with high levels of hypochlorous acid (more than 500 ppm), the aqueous hypochlorous acid composition having low chloride concentrations (maximum chloride levels of 1:3 chloride to hydrochlorous acid) and a pH between 3.5 and 7.0 to stabilise the composition without the need for additional stabilisers. A solid composition is also provided for producing the stable solution.

The present invention relates to stabilised hypochlorous acid (HOCl), processes for its production and various applications for the use of the composition.

The use of sterilising and disinfecting solutions to remove bacterial, viral or fungal contamination is well established in the art. Sterilising solutions, disinfectants and bleaches, are routinely used to provide a sterile or sanitised environment. Such a use is particularly important in environments such as medical, veterinary, agricultural, food processing and dental where, for example, patients and/or the environment is susceptible to infection and cross-infection. Pathogen destruction is required where equipment is used for surgical procedures or other intervention of the human or animal body, as well as in crop management, fresh food production and water treatment.

It will be appreciated that the provision of sterile and sanitised environments and equipment has become more important as a result of the increasing incidence of hospital borne general environmental infections, such as Methicillin-resistant Staphylococcus aureus (MRSA) and Clostridium difficile.

Hypochlorous (or hydrochlorous) acid (HOCl) is already recognised in the art as an effective antimicrobial agent with activity against bacteria, viruses, fungi and spores. It is particularly favoured for use in medical, veterinary, agricultural and industrial uses as it is free from oral toxicity, skin sensitisation or irritation (including eye irritation) and is non-mutagenic.

However, there are a number of problems associated with the use of hypochlorous acid. In particular, solutions of HOCl are very unstable, with a short half-life of around 48 hours. Therefore, the solutions rapidly lose their anti-microbial activity and hence, their effectiveness. The current solution to this problem is to prepare the HOCl solutions in situ as required, using the electrolysis of a solution of brine. This requires on-site apparatus to prepare the solution, with the resulting high cost associated with both the preparation of the HOCl solution and the inactive waste solution associated with its short half life. Electrolysis is also a slow and expensive process which is open to variation and often difficult, if not impossible, to validate.

Oxidising agents such as chlorine, bromine and chlorine dioxide are widely used as highly efficacious antimicrobials however a disadvantage is the long term storage stability in aqueous solutions. In the case of chlorine and bromine, the pH also has a major influence on the prevalent active species that impacts both the efficacy and bleaching properties.

Several methods have been used to improve storage stability via the addition of halogen stabilisers and the use of pH buffers.

U.S. Pat. No. 2,438,781 (Mar. 30, 1948) relates to the stabilisation of hypochlorite solutions and cites products suitable for use as stabilising agents in solutions of alkali metal hypochlorite's, thus providing extended shelf life. Benzene N sodium-sulphonamide was demonstrated to provide the greatest stability on the basis of weight. Such stabilised halogen forms generate a range of combined halogens with various degrees of microbial efficacy rather than a clearly defined microbiocidal species.

For the control of micro-organisms and bleaching the use of buffers to stabilise solutions of N-Halo compounds is described in U.S. Pat. No. 3,749,672 (Jul. 31, 1973) where aqueous solutions having a pH between 4 and 11 are prepared by adding a hypochlorite to certain N-hydrogen compounds such as sulfamic acid. The buffers stabilise the pH between 4 and 11 and in alkali pH the predominant species is hypochlorite anion rather than highly efficacious Hypochlorous acid.

U.S. Pat. No. 6,162,371 (Dec. 19, 2000), is directed towards an aqueous solution of a source of unipositive chloride ion, a chlorine stabilising agent, an acidic buffer to stabilise the pH from 2.0 to 6.5 wherein the chlorine stabiliser and the source of the source of unipositive chlorine ion are in a molar ratio of greater than 1:1. This composition however may evolve chorine gas in acidic conditions according to Raman spectroscopy (D Cherney et al, 2006) and no attempt is made to control chlorite levels which are intrinsically high due to the use of sodium hypochlorite.

Another way to improve shelf life is to form dry state compositions as described in WO/1991/003936 where a composition comprising a water soluble inorganic halide, a strong oxidising agent which, in aqueous solution, reacts with the halide to generate hypohalite ions and sufficient sulphamic acid which acts as a halogen acceptor. A water soluble carbonate or bicarbonate reacts with excess sulphamic acid to produce carbon dioxide for effervescence. The aqueous solutions however are not claimed to be stable long term.

N—hydrogen compounds such as 5,5 dimethylhydantoin and their chlorinated derivatives have demonstrated improved stability compared to unstabilised halogens and such technology is widely in use in areas such as slime control in paper mills. The combined forms and intermediates such as monochlor hydantoin release halogen upon demand rather than being based on a ready to use high purity hypochlorous acid solution. U.S. Pat. No. 6,471,974 (Jun. 29, 1999) relates to N-chlorosulfamate compositions having enhanced microbial activity via buffering and the use of enhancing dopants such as 5,5 dimethylhydantoin which results in a range of halogen species which is undesirable from an end user and regulatory perspective. Additionally, the low pH may result in the generation of Chlorine gas and there is no attempt to minimise the chloride ion content.

Hypochlorous acid (HOCl) is a weak acid that exists when Chlorine is dissolved in water. In aqueous solution, HOCl partially dissociates into the anion hypochlorite OCl—

Cl₂+H₂O<------>HOCl+H⁺+Cl⁻

HOCl<------>H⁺+OCl⁻

At pH 7.5 equal concentrations of HOCl and OCl− exist, above this pH the predominant species is OCl− with total ionisation occurring at pH 9.5.

HOCl has been identified as the principle biocidal active species. Therefore, the biocidal efficacy decreases in the more alkaline environments due to the decreased level of HOCl (Rideal and Evans 1921) and Johns (1934).

It is possible to produce HOCl from chlorine gas, liquid sodium or lithium hypochlorite, or from the powders calcium hypochlorite, sodium dichloroisocyanurate dehydrate and trichlorocyanuric acid. What mostly exists in sodium and calcium hypochlorite solutions is the hypochlorite ion, which is up to 120 times less effective than hypochlorous acid as a disinfectant as demonstrated by Kapoor, S. K, (1968)

The small molecular size of HOCl coupled with an electrically neutral charge enables rapid penetration into microbial cells where the HOCl quickly inactivates the viability of microbial cells via inhibition of enzymes (Knox et al, 1948), intracellular ATP hydrolysis (Barrette et al, 1987), inhibition of DNA replication (Rosen et al, 1998) and the induction of protein aggregation (J Winter et al, 2008).

HOCl sporicidal activity has also been well documented (Rudolf and Levine 1941) where 25 ppm available chlorine solutions were applied at a range of pH levels and the time taken to produce a 99% kill of B. metiens spores was determined. The results show 2.5 mins for pH 6, 3.6 mins for pH 7, 5 mins for pH 8, 19.5 mins for pH 9, 35.5 minutes for pH 9.35, 131 minutes for pH 10 and 465 minutes for pH 12.86.

As mentioned above, HOCl has been generated electrochemically but the major drawbacks of this include its lack of stability and pH maintenance requiring on site generators. Typically there are also high chloride residuals due to a very high brine content which can lead to residues and increased corrosion.

US Patent Application Publication US 2009/0258083 A1 (Oct. 15, 2009) describes a method for preparing a stabilised antimicrobial hypochlorous acid solution by diluting an aged stock to provide HCl solutions at 50 to 7000 ppm at a pH range of 2.8 to 4.0. At these pH levels there is the likelihood of Chlorine generation and the high chloride levels from the sodium hypochlorite and hydrochloric acid are undesirable as regards stability, corrosion, purity and residual.

The unpublished U.K. Application No. 1021287.6 (Mallet, C et al.,) describes the preparation of a stable HOCl solution comprising an aqueous solution of hypochlorous acid and a salt MₙX_{y}, where M is hydrogen or a metal and X is a conjugate base of an acid having a pKa of from 4 to 7 and where n and y are independently 1, 2 or 3 and the salt MₙX_{y} has an overall neutral charge, said composition having a pH from 4 to 7. This composition has been shown to retain its antimicrobial activity for many months by maintaining the pH of the solution within a particular range. However, it does require the inclusion of a salt within the composition which may not always be desirable. It would also be preferable to be able to produce the stable solution without the need for halogen stabilisers, such as sulphamic acid.

It is an aim of the present invention to provide a stable solution of HOCl which maintains its activity over a longer period of time.

It is a further aim of the present invention to provide a more stable solution of HOCl which retains its microbiocidal activity during standard processing procedures, such as bottling and other industrial processing and that may be subjected to reasonable variations in environmental conditions without degradation thereby enabling it to be packaged, bottled, transported and stored for a longer period of time prior to use.

Yet a further aim of the present invention is to provide a more stable solution of HOCl that may be produced rapidly, in large volumes and more cheaply than the prior art process of electrolysis.

Another aim of the present invention is to provide a HOCl based antimicrobial aqueous solution with a mild pH that has minimal chloride content and does not require the inclusion of stabilisers.

Accordingly, a first aspect of the present invention provides a composition comprising a stable solution of hydrochlorous acid wherein the chloride to hydrochlorous acid ratio is within the order of 1:3, more preferably at least 1:5, more preferably 1:10, and the pH of the solution is maintained at a pH of between 3.5 to 7.0 whereby the stable HOCl retains its antimicrobial activity for a period of at least one month, preferably at least three months, especially at least six months.

It has been found that buffering the solution to within the pH range 3.5 to 7 enables the HOCl to remain stable in the solution for significantly longer periods of time, in excess of three months, indeed normally in excess of six months. The solution can also have much higher activities than the prior art, in excess of 600 ppm.

HOCl is at maximum concentration in solution when the pH is between 5.5 and 6.5. In a preferred embodiment of the first aspect of the present invention, the composition has a pH from 5 to 6.8, more preferably from 5.5 to 6.5, especially 6 to 6.5.

The level of Hypochlorous acid can be defined and controlled for specific application requirements between 10 and 130,000 parts per million, more preferably between 60 to 50,000 parts per million.

For the purposes of the first aspect of this invention, the halogen source for the HOCl is preferably a calcium salt, such as calcium hypochlorite, wherein the calcium has been removed from the solution. However, other halogen sources may be used. HOCl partially dissociates in aqueous solution to form a hypochlorite anion, OCl⁻. It is not necessary to demonstrate the chemistry at this point as it is common.

HClO [symbol] OCl⁻+H⁺

The degree of dissociation will depend on the pH of the solution. Without being bound by scientific theory, it is understood that the active species in the composition of the present application is HOCl. The provision of a solution with low chloride levels results in greater amounts of the active HOCl species in the solution.

The aqueous solution of HOCl is prepared by the addition of a source of chlorine to water to obtain a solution having a concentration of chlorine of concentrations from 1 ppm to 10,0000,000 ppm. Ideally, deionised water is used as the solvent.

The composition of the first aspect may optionally comprise additional components. In particular, an acid or alkali may be added to the composition to maintain the pH of the solution between the critical pH range of 3.5 to 7. Examples include phosphoric acid or sodium hydroxide.

The composition according to the invention may comprise other additional components to make it more suitable for its intended use. Such additional components include, but are not limited to, moisturising agents (such as Moisturiser Surfacare ARM HE), surfactants, fragrances, emollients, chelating agents, colourants, optical enhancers, additional biocides or adjuvants and/or chlorite donors. It is to be appreciated that the additional components should be halogen stable, having no halogen demand, and should not oxidise the HOCl, for example by removal of the chloride.

The composition may also include a halogen releaser, such as dimethyl hydantion, in order to provide a more controlled release of the halogen. An equilibrium stabiliser, such as sodium chlorate, may also be included to prolong storage stability.

The composition according to the first aspect of the present invention exhibits improved stability when compared with known electrolytically prepared solutions of HOCl. In particular, the composition is sufficiently stable that it can be packaged, stored and transported. This avoids the current requirement to prepare HOCl on demand, thereby providing HOCl in a more convenient and highly efficacious form. As a result of the improved stability, there is no requirement for the HOCl to be prepared in situ. The solution of the present invention is therefore, not prepared by electrolysis.

A second aspect of the present invention provides a process for producing a stable aqueous composition of hypochlorous acid, the process comprising the steps of adding a source of chlorine to water, manipulating the chloride levels to be at a maximum of 1:3 chloride:hypochlorous acid, and controlling the pH of the solution to between 3.5 and 7. More preferably, the ratio is 1:5, more preferably still 1:8, especially 1:10 chloride:hypochlorous acid.

In the context of this disclosure, “stable” means a composition that retains its antimicrobial activity for a period of at least 3 months, more preferably, at least 6 months when stored in sealed containers at ambient temperature.

Examples of suitable non-oxidising acids with a pKa of from 5 to 6.8 for controlling the pH of the solution include an organic acid such as acetic acid, N-(2-acetamido)-2-iminodiacetic acid (ADA), benzoic acid, 1,3-bis[tris(hydroxymethyl)methylamino]propane (BIS-TRIS propane), carbonic acid, citrate, 2-(N-morpholino)ethanesulphonic acid (MES), piperazine-N—N′-bis(2-ethanesulphonic acid) (PIPES), succinic acid, formic acid, lactic acid, carbonic acid, tartaric acid, benzoic acid, phosphoric acid, phosphorous acid, oxalic acid, boric acid, maleic acid, adipac acid, citric acid. Preferred examples include one or more of citric acid, adipac acid or phosphoric acid. The acid can also be a mineral acid such as HCl or HNO₃.

It will be appreciated that the amount of acid to be added will vary depending on the initial pH of the solution and the concentration of the acid. Any concentration of acid can be used to acidify the HOCl solution, for example 10 mM, 100 mM, 1M or 10M acid solutions can be used. The acid may be added in a batchwise, or dropwise manner and the pH of the solution is preferably monitored. The acid is added until the pH of the solution is in the required range, i.e. from pH 3.5 to 7.

If excess acid is added to the solution, thereby depressing the pH of the solution to below pH 3.5, base should be added to the solution to increase the pH of the solution to between 3.5 and 7. Examples of such bases include caustic soda and similar alkalines.

A foaming form of the composition may be provided according to the present invention. To this end, a third aspect of the present invention provides a composition according to the first aspect additionally comprising a surfactant. Surfactant causes a foaming of the sanitiser which may be beneficial for certain applications, for example for holding HOCl to the skin, such as hands, for a prolonged period.

The amount of surfactant provided in the third aspect of the present invention will depend upon the intended application of the composition as discussed below. For the purposes of this invention, the surfactant is present in an amount of 0.5 to 2.5% by volume. Preferably, the surfactant can be present in an amount of 1 to 1.5% by volume. The surfactant must have no halogen demand (i.e. they must be chlorine stable) and must not oxidise HOCl. Examples of suitable surfactants include N-methyl-N-(1-oxododecyl)-glycine sodium salt, N-alkyl “tallow” N,N-bishydroxyethyl amine oxide, lauramine oxide, Surfac A030 (Ammonyx Lo, C12-C18alkyldimethyl N-oxides CAS NO 68955-55-5 EINECS 273-2,8-2), myristyl amine oxide, sodium lauroyl sarcosinate, PEG-7 glyceryl cocoate, N′N-dimethyltetradecylamine N-oxide, and lauryl ether sulphates such as Steol CS230KE AES96 (sodium laureth sulfate). Preferably, the surfactant is an amine oxide but other surfactants may be used.

A fourth aspect of the present invention provides a process for preparing an aqueous solution of hypochlorous acid having low chloride levels, the process comprising the adding solid calcium hypochlorite and at least one phosphate buffer to deionised water. The components may be added directly or via a filtering device, such as a perforated bag that is added to water.

Preferably, ultra low chloride calcium hypochlorite is used in the process to provide the required low chloride levels. More preferably, at least 70% assay pure calcium hypochlorite is used, more preferably still even purer calcium hypochlorite is used.

In a more preferred process of the present invention, the process comprises the steps of:

adding chlorine to calcium hydroxide to provide a solution of calcium hypochlorite, calcium chloride and water;

removing the calcium chloride in solution to provide calcium hypochlorite;

dissolving the calcium hypochlorite in water to provide an alkaline solution of calcium and hypochlorite ions;

precipitating calcium as calcium hydroxide to enable its subsequent removal;

adjusting the pH of the resultant solution of hypochlorite ions to around 5-6 to precipitate any remaining calcium to enable its subsequent removal; and

adjusting the pH of the solution of hypochlorous acid to a pH of between 3.5 and 7.

The pH is adjusted by means of an appropriate acid or alkali, such as phosphoric acid or sodium hydroxide. The use of calcium hypochlorite source enables reduced chloride levels to be achieved than with other chlorine sources, such as sodium hypochlorite. For example, 5000 ppm of HOCl solution typically contains less than 500 ppm chloride when made with calcium hypochlorite compared to approximately 2500 ppm if sodium hypochlorite was used.

The process according to the fourth aspect of the present invention provides a novel route to generate high purity, long term stable HOCl solutions without the need for stabilisers via the control of the chloride content, removal of the calcium and pH optimisation. The pH of the resulting solution is preferably maintained at a pH of 3.5 to 7.0 to optimise the level of HOCl and prevent the generation of chlorine, more preferably between 5.5 and 6.5. The low chloride level, lack of additional stabilisers and pH range of the composition formed according to the process are desirable handling and applications attributes.

Preferably, the calcium hydroxide and calcium phosphate precipitate is removed by filtration.

A second chlorination stage may be included in the process to enhance the hypochlorite concentration, involving the re-dispersion of solids from the previous stage and reacting it with more chlorine, followed by subsequent removal of calcium chloride.

A fifth aspect of the present invention is directed to an aqueous solution of hypochlorous acid prepared according to the process of the fourth aspect of the present invention.

The solution according to the first, third and fifth aspects of the present invention may be diluted as appropriate. Dilutions should be carried out with deionised water to prevent contamination with chloride ions. Additional components may be added to the composition as required. For example, magnesium sulphate may be included for medical benefit.

Selective crystallisation of sodium hypochlorite or a combination of electrochemical methods and membrane technology may be used as alternative routes to generate the low chloride HOCl solution according to the present invention.

A sixth aspect of the present invention provides a solid composition for producing a stable solution of hydrochlorous acid according to the first aspect of the present invention, the composition comprising a solid halogen source and a separate solid acid, being encapsulated together or separately by a non-halogen demanding coating.

The encapsulated solid composition according to the sixth aspect of the present invention prevents the halogen source and acid from reacting to form the hydrochlorous acid until contact with water or other aqueous solution. More preferably, the composition is ahydrous.

The halogen source ideally comprises ultra low chloride calcium hypochlorite, preferably being at least 70%, more preferably at (east 75% assay pure calcium hypochlorite. The solid acid preferably comprises citric or adipic acid but is not limited thereto. For example, other acids that may be used include, but are not limited to di- or tri-carboxylic acids, such as oxalic, malonic, succinic, glutaric, pimelic and suberic acids. Anhydrous phosphoric acid may also be used but does have the limitation of a low melting point of 42° C., making it unsuitable for use in warm climates'.

Any suitable encapsulation process known in the art may be used to encapsulate the halogen source and the acid. The halogen source and the acid may be encapsulated together within a single coating or separately encapsulated. Preferably, a water-soluble coating, such as gelatin or gelatin substitutes, is used. The encapsulation coating may be a water soluble polymer which may be selected based on whether immediate release of the active ingredients, i.e. the halogen and acid sources, is required on contact with water or a delayed (i.e. time dependent) release is required. It is to be appreciated that any standard method of encapsulation may be used, such as air suspension methods, centrifugal extrusion, vibrational nozzle, spray-drying, ionotropic gelation, coacervation, interfacial poly condensation or cross linking or in-situ or matrix polymerisation.

In a further preferred embodiment of the sixth aspect of the present invention the active ingredients are encapsulated within shells or exines obtained from the spores of plants using a technology developed by Sporomex Limited of Hull, U.K. Plant pollens or spores from species such as Lycopodium have their internal genetic material removed by a chemical or enzyme process to leave a porous, inert, elastic shell called the exine. The active ingredients are migrated into the spores which are then protected until later release. When mixed together with water, the spores release their contents enabling the acid and hypochlorite to mix to create HOCl at a given pH and ppm. The shells are particularly beneficial as they can withstand alkali and acid treatment, as well as temperatures of at least 250° C. The relatively large central void within the shell means high loading levels can be achieved, such as 4:1 (active ingredient:exine, w:w) for a 40 micron diameter particle.

Further details regarding the process used for the preparation of such spores is described in PCT Publication No. WO2005/000280 in the name of The University of Hull. In brief, the genetic material is removed through the permeable exine and then the active ingredients are allowed to migrate through the pores, optionally with the aid of a filling enhancing agent such as ethanol or a physical aid, such as a vacuum.

Preferably, the solution formed from the powders of hypochlorite and acid is filtered, more preferably through a flexible fine filter (eg 1-5 micron diameter holes) to trap calcium, calcium citrate, calcium hydroxide or any other debris resulting from the chemical reaction.

The encapsulated solid ingredients may be surrounded by a fine filter to retain any solid deposits, debris, exines etc following formation of the hydrochlorous acid.

It is to be appreciated that the anhydrous version of the composition according to the sixth aspect of the present invention may be made into any appropriate solid shape, such as tablet form, powder or briquette. The ratio of hypochlorite:acid contained within the composition will determine the ppm of hydrochlorous acid produced in a particular volume at a given pH thereby enabling the concentration of the composition to be tailored for a particular application.

The stable nature of the HOCl compositions of the present invention enable the compositions to be provided in a variety of different formats for their appropriate delivery to a site for treatment. For example, these may include, but are not limited to wipes, lotions, gels, powders and foams. Methods of delivery of the composition of the invention may also vary but includes pouring, injection, pumping, immersing, coating, spraying, misting and fogging.

The various stable HOCl compositions provided according to the present invention may be used in a wide range of applications. Examples of such applications are detailed below but it is to be appreciated that the use of the invention is not limited thereto.

The compositions are particularly suitable for sanitizing or sterilising hard surfaces, living organisms and environments generally. In particular, a primary use of the composition is as a antimicrobial agent. The composition may be used as an antibacterial agent, an antiviral agent, an antifungal agent or an antisporal agent. The composition may act as a microbiocidal agent (i.e. to kill the micro-organisms) or as a microbiostatic agent (to inhibit or prevent the growth of the micro-organisms). The composition will either completely eradicate the bacteria, viruses, fungi and/or spores or may reduce the number or activity of bacteria, viruses, fungi and/or spores (particularly pathogenic bacteria, viruses, fungi and/or spores) so that they no longer have a detrimental effect.

The composition is particularly provided for use as an agent against one or more of Clostridium difficile including variant 027, Staphylococcus aureus, Pseudomonas aeruginosa, Enterococcus hirae, Candida albicans, Aspergillus niger, Escherichia coli, Klebsiella pneumoniae or Acinetobacter sp.

The composition may be provided for use in medicine (i.e. to treat infection in a patient) or to sterilise equipment or an environment. In particular, the composition may be used to kill bacteria, viruses, fungi and/or spores on surfaces or equipment, for example in homes, medical institutions such as hospitals, doctors' surgeries, veterinary surgeries or dental surgeries or work places. The composition is particularly suitable for domestic use, for example to sterilise bathrooms and/or kitchens in particular surfaces used for the preparation of food such as work tops etc, or to sterilise equipment used for the preparation of food, such as chopping boards, knives, baby bottles, etc.

The composition may also be used for the sterilisation of medical instruments, including those used for medical, dental or veterinary uses. The composition may be used to sterilise instruments which contact the body such as endoscopes, clamps, sutures, scalpels etc, especially those that are difficult to sterilise such as those which cannot be autoclaved. To this end, a sixth aspect of the present invention provides a method of sterilising an object comprising contacting the object with the composition of the first, third or fifth aspects of the invention. For the purpose of the invention, the object may be immersed in the composition or the composition may be applied to the object by spraying, misting etc or using an applicator such as a cloth, brush, swab or mop.

The composition may be used in medicine. The composition is particularly provided for the treatment of bacterial, viral, fungal or sporal infections on the external surface of the body (i.e. on the skin or in the mouth). The composition is therefore, particularly provided for topical administration. The composition has been found to be suitable for treating wounds, cuts or ulcers. The composition may be applied directly to the wound and aids in the healing of the wound by sterilising the wound (i.e. by killing microorganisms of all types). In particular, the composition may be provided as a wound irrigant. The composition may be used to treat burns, post-surgical wounds and ulcers, such as static ulcers, pressure ulcers, chronic ulcers, venous ulcers and diabetic ulcers. Other medical conditions both in humans as well as animals may also be treated where pathogens are present and are the target.

Where the composition of the first, third or fourth aspects is provided for such a medical use, the pH of the solution can be adjusted to avoid any irritation or discomfort to the patient. The pH of the solution can be adjusted to pH 5 to 6, preferably pH 5.4 to 5.8 to avoid such irritation.

Accordingly, the composition of the present invention may be used for treating topical microbial infections comprising topically applying the composition to the site of the infection. It will be appreciated that the composition may be applied directly or be impregnated onto a pad, swab or bandage, a cream or gel which is applied to the site of infection. The composition may be applied once or repeatedly over a period of time necessary to allow the antimicrobial infection to be treated.

It is not possible to impregnate a cellulose paper or pad with the composition according to the present invention. It is necessary to impregnate a non-woven material with the composition. To this end, a seventh aspect of the present invention provides a non-woven material impregnated with a composition according to the first, third or fourth aspects of the present invention.

Preferred wipes are polypropylene non-woven wipes. More preferably, a halogen stabiliser is included with this aspect of the composition.

An additional application for the compositions of the present invention is for sterilising a foodstuff. Foodstuff such as meats, fish, dairy products, vegetables or fruits. Treatment of the foodstuff with the composition has been found to significantly increase the shelf life of the foodstuff and decrease the risk of contamination. For vegetables such as salad vegetables, the vegetables may be contacted with the composition to improve the crispness, shelf life and appearance of the vegetables. It will be appreciated that the composition of the present invention may act as an antimicrobial agent against micro-organisms which cause disease such as food poisoning, salmonella, listeria etc. Alternatively and/or in addition the composition may act as an antimicrobial agent against microorganisms responsible for food spoilage. For meats such as cut chicken, beef, lamb and venison, contact with the composition can significantly improve their shelf life, tenderness and appearance.

The composition of the invention may also be used in sterilising liquids such as water. The composition is particularly provided for sterilising drinking water. Alternatively, the composition can be used to sterilise water in swimming pools, ornamental pools, fountains etc. In a preferred embodiment the composition may be dosed into water break tanks at a predetermined rate. Alternatively, the composition may be added at source.

Another important application for the present invention relates to ice comprising a composition of the first, third or fourth aspects of the invention. To this end, an eighth aspect of the invention comprises a composition of the first, third or fourth aspects frozen to provide ice. The ice may be used in a number of medical and commercial ways, including but not limited to, the storage of organs for transplant or other biological tissue, and the preservation of foodstuffs for example fish including seafood, meat, vegetables or dairy products.

Forms of HOCl produced by electrolysis are difficult to freeze due to the brine solution from which they are produced. The composition of the invention is produced without brine is therefore ideal for the creation of ice.

A further application for the composition of the present invention comprises a laundry sanitiser. Laundry and clothing are a breeding ground for many bacteria, viruses and pathogens. Their sanitisation is therefore critical within the consumer, medical and industrial arenas. At present, heating the wash water to temperatures above 60° C. is predominantly used to sanitise laundry. This heating requires substantial energy and in many instances does not provide the desired effect as many pathogens have become resistant to higher wash temperatures. The composition of the invention provides a laundry sanitiser for the sterilisation of fabrics, including but not limited to. clothing, sheets, mops, towels, carpets, mats, shoes, hats, gloves and other items washed in a household, medical, veterinary, dental or industrial washing machine or hand washer. For the purpose of the invention, an aqueous or anhydrous composition of the invention will be dispensed into the wash during the wash cycle. In a preferred embodiment of the invention the composition will be added to the wash during the final rinse cycle after the laundry has been washed. In an alternative embodiment the composition may include surfactants capable of both cleaning and sanitising the laundry as well as moisturisers and conditioners.

Yet a further application for the composition of the present invention is as a sanitiser for sensitive areas of the body. Sanitising to a high log reduction (greater than log 6) of the lips, inner and outer ear, mouth, teeth, tongue, feet, under arms and genitals is presently difficult or impossible with the existing prior art. Many of the prior art sanitisers are unsuitable or unsafe for the use on sensitive areas of the body. A composition of the invention would therefore be highly suited use in these areas. The composition of the invention provides a sensitive area sanitiser for the sterilisation of the afore mentioned areas.

The composition of the present invention may also be used as a deodoriser. Smells are often associated with airborne bacteria which can be difficult to eradicate and control odour in rooms, fridges, lorries, cupboards, kitchens, industrial plants, areas or open spaces. As a highly active microbiocide the composition of the invention may be sprayed, misted or fogged into an area to eradicate and control odour. This may be done continuously or as and when necessary. In a preferred embodiment of the invention a composition of between 100 ppm and 5000 ppm would be used in this application.

Yet a further application for the present application is use of the composition on cut flowers to increase their life. Bacteria and other microbes are often the cause of wilting or degradation of flowers. A composition of the invention may be added to the water in which the flowers are held to destroy bacteria and other pathogens and thus extend the life of the flowers and to reduce levels of cross contamination at growing source and in packaging. Alternatively, the composition may be sprayed over the flower, or the stems or heads could be dunked in the composition. In a preferred embodiment for the invention a composition of between 1 ppm and 100 ppm would be used in this application.

Furthermore, the stable HOCl composition according to the present invention may be used as a replacement or supplement to antibiotics in cases such as gut, bowel, and colon treatment but not restricted to the treatment of such conditions. The invention is a very strong oxidising agent as previously mentioned and as such destroys the DNA and RNA of pathogenic cells. Therefore, no immunity can be built up by subsequent pathogens as is often the case experienced in pathogens that have been treated by way of antibiotics. The non-toxic nature of the invention also means that it may be applied as a sterilant within the realms of mammalian surgical procedures such as in root canal dentistry or where antibiotics would be the normal course of further preventive measures.

The composition of the invention may be applied in both pre- and post-operative surgical protocols. Patients bound for surgery may be exposed to the invention by way of spraying, fogging or by wash methods. This protocol procedure would minimise the risk of cross contamination in a theatre environment. It is intended that the invention is fogged or sprayed onto the open wound area during the surgical procedure and as sewing up or other methods are employed thus further minimising the ingress of harmful pathogens both in limb and body cavity areas.

The invention provides a hand or skin sanitiser comprising a composition of the invention. The hand sanitiser is provided to act as a microbicidal or as a microbiostatic on the surface of the hands or skin thereby either completely eradicating the bacteria, viruses, fungus and/or spores or reducing the number or activity of bacteria, viruses, fungus and/or spores (particularly pathogenic bacteria, viruses or fungus) so that they no longer have a detrimental effect. Preferably, the hand or skin sanitiser has a surfactant at levels of 0.1 to 2.5%, so areas of soil that require a degree of degreasing in order for the sanitising process to be highly efficacious are addressed.

Surfactant also causes a foaming of the sanitiser which holds HOCl to the hands for a prolonged period. This increases the efficacy and prevents the solution falling from the hands a problem associated with the present art. Best practices and regulations require a volume of 3 ml-5 ml of fluid when sanitising the hands. Standard solutions of HOCl of this volume would result in considerable liquid loss to the floor and clothing, an undesirable effect. The foaming action of the surfactant prevents this and ensures there is sufficient HOCl solution to ensure cleaning of the palms, back and fingers.

Where the composition of the first aspect is provided as a hand or skin sanitiser, the composition will preferably comprise a moisturiser at a level of 0.1 to 1.0% by volume. In this embodiment, the volume of surfactant is preferably 1 to 1.5% by volume.

Where the composition of the first aspect is provided for such a use, the pH of the solution can be adjusted to avoid any irritation or discomfort to the user. The pH of the solution can be adjusted to pH 5 to 6, preferably pH 5.4 to 5.8 to avoid such irritation.

The invention may be in the form of a disinfectant surface cleaner. The surface cleaner is particularly provided for the disinfection of hard surfaces in household, medical, veterinary, dental and industrial environments. For the purpose of the invention, the cleaner can be applied directly to the surface by spraying, misting etc or using an applicator such as a cloth, brush, swab or mop. This embodiment preferably contains a surfactant at levels of 0.1 to 2.5%, so areas of soil that require a degree of degreasing in order for the sanitising process to be highly efficacious, are addressed.

The invention may also be used for the treatment of food crops such as wheat, barley, maize, oats, salads, soft fruits, vegetables as examples where the control of spores, viruses, bacteria, moulds and fungi is vital to ensure health crops with increase yields. Agrochemical companies across the globe spend huge sums of money on developing new biocides for use in farming but in many cases the target pathogens build up a natural immunity to these biocides over a period of time. This aspect of the invention will allow farmers to spray crops in a more efficient manner by reducing the surface tension of the target plant and allowing the HOC) to adhere more efficiently thus providing for a greater reduction all of the aforementioned pathogens whilst allowing for immunity problems associated with the current biocides not to be an issue.

The invention may also be used for the disinfection of poultry, dairy cattle, pigs, goats, sheep and other animal housing, farmyards, milking parlours, standing areas without the resultant corrosion and dangerous gases associated with current products on the market such as hypochlorites and quat ammoniums as examples.

Further potential uses of the composition according to the invention include, but are not limited to:
the treatment via oxidation of cancer cells thus enhancing the immunogenicity and stimulation.
the treatment of sunburn, sun damaged skin, freckles, liver spots, warts, verrucas and acne.
the treatment of ageing skin and damaged skin.
the treatment of scars and scar tissue.
the treatment of psoriasis and eczema, cold sores, athlete's foot, spots and skin ulcers, mouth ulcers, gingivitis, and general periodontal health.
use as a general slimicide and a pesticide.
the reduction and elimination of airborne pathogens via fogging, and the treatment of human and animal lung disorders including tuberculosis.

The invention will now be illustrated by way of example only to the following Examples in which Example 1 describes the preparation of a stable HOCl solution having low chloride levels according to a first aspect of the present invention and assesses its stability over a period of one month and Example 2 describes the preparation of a stable HOCl solution prepared from encapsulated powdered calcium hypochlorite and citric acid monohydrate; and with reference to the accompanying drawings in which:

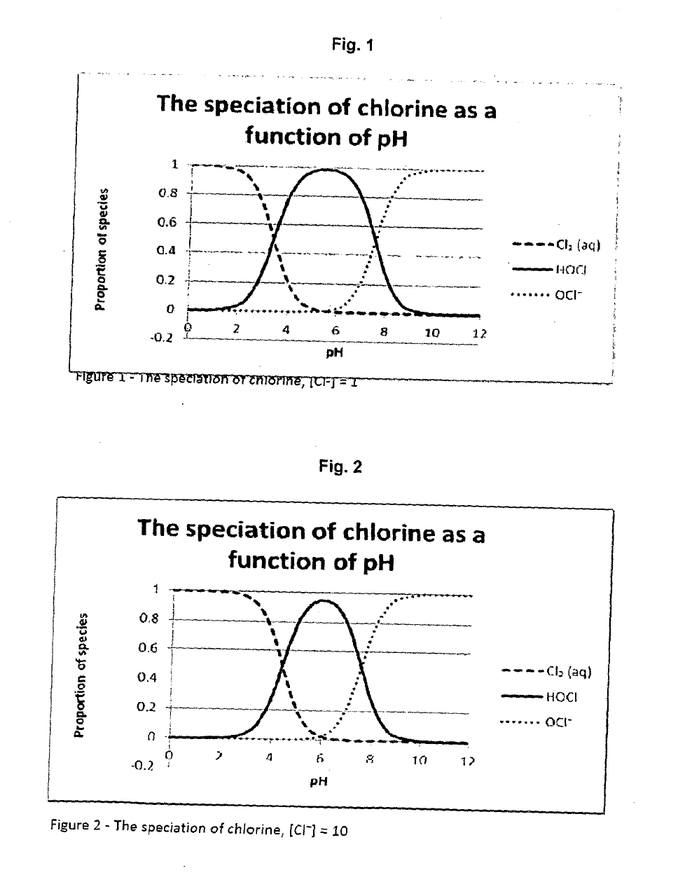

FIG. 1 is a distribution curve of chlorine species against pH showing the distribution of chlorine species as a function of pH;

FIG. 2 is a distribution curve having the chloride concentration increased by a factor of 10; and

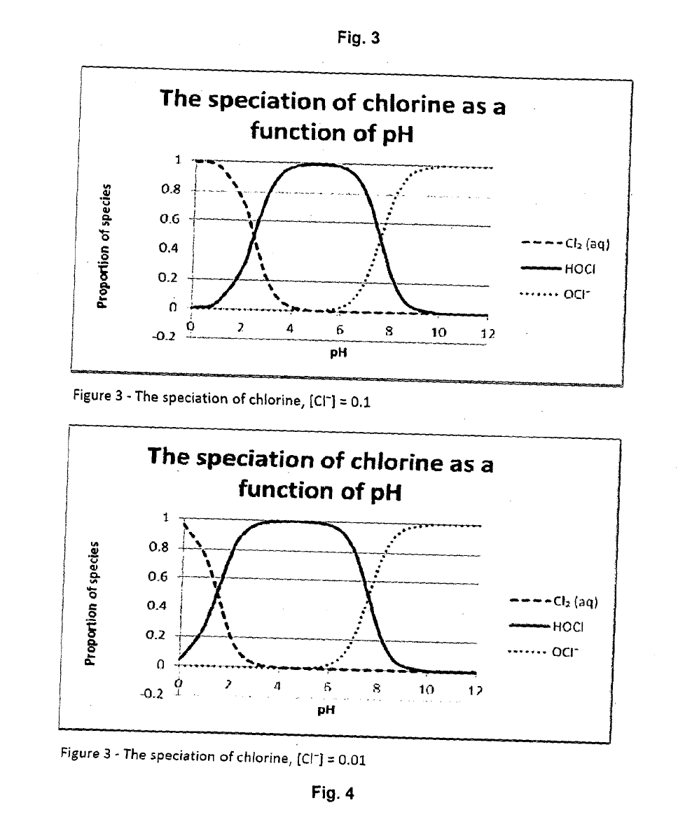

FIGS. 3 and 4 are distribution curves illustrating the affect of lowering chloride content on the distribution of the chlorine species.

### EXAMPLE 1

### Investigation into a Stable HOCl Composition Having Low Chloride Levels According to the Present Invention

The present invention relates to the true stabilisation of hypochlorous acid. Many have proposed the use of additives that combine with hypochlorite or hypochlorous acid to form distinct compounds or complexes. These materials are said to stabilise the solution. The solution is therefore not pure hypochlorous acid, but an equilibrium of hypochlorous acid with the additive.

Often a high percentage of the chlorine is present as combined chlorine rather than free chlorine, where the free chlorine represents the total un-combined hypochlorous acid and hypochlorite. For some additives the combined chlorine species have a slight microbiological activity, for others they do not.

There are obviously consequences to adding other components; simply increasing the dissolved solid content may be detrimental in certain applications; some additives are harmful, precluding their use where the water contacts food or food contact surfaces; some are unacceptable if the water is for drinking, for medical applications or agricultural use. It is therefore desirable to have a pure hypochlorous acid solution or one that has only very low levels of an additive.

This invention has a theoretical basis which has been confirmed by spectroscopic analysis, laboratory work and small scale manufacturing.

Chlorine hydrolyses rapidly in water to form hydrochloric acid and hypochlorous acid and hypochlorite as follows.

Cl₂+H₂O<------>HOCl+H⁺+Cl⁻

HOCl<------>H⁺+OCl⁻

The equilibrium constant for these reactions are represented by:—

$$\begin{matrix}{K}_{1} = \frac{[ HOCl ] [ {H}^{+} ] [ {Cl}^{-} ]}{[ {Cl}_{2} ]} & ( 1 ) \\ {K}_{2} = \frac{[ {H}^{+} [ {OCl}^{-} ]}{[ HOCl ]} & ( 2 )\end{matrix}$$

The relative proportions of CL₂, HOCl and OCL⁻ at some fixed concentration of Cl⁻ can be expressed as:—

$$\begin{matrix}{Cl}_{2 } = \frac{[ {Cl}_{2} ]}{[ {Cl}_{2 ]} ] + [ HOCl ] + [ {OCl}^{-} ]} & ( 3 ) \\ HOCL = \frac{[ HOCl ]}{[ {Cl}_{2 ]} ] + [ HOCl ] + [ {OCl}^{-} ]} & ( 4 ) \\ {OCl}^{-} = \frac{[ {Cl}_{2} ]}{[ {Cl}_{2 ]} ] + [ HOCl ] + [ {OCl}^{-} ]} & ( 5 )\end{matrix}$$

Using the expressions for the equilibrium constants in Eq (1) and Eq (2) and the relationship that pH is equivalent to the negative logarithm of the hydrogen ion concentration we get the following expressions:—

$$\begin{matrix}{Cl}_{2} = \frac{1}{1 + {K}_{1} / [ {Cl}^{-} ] \times {10}^{p H} + ( {K}_{1} {K}_{2} ) / [ {Cl}^{-} ] \times {10}^{2 p H}} & ( 6 ) \\ HOCl = \frac{1}{1 + ( [ {Cl}^{-} ] / {K}_{1} \times {10}^{p H} ) + ( {K}_{2} \times {10}^{p H} )} & ( 7 ) \\ {OCl}^{-} = \frac{1}{1 + ( [ {Cl}^{-} ] / {K}_{1} {K}_{2} \times {10}^{2 p H ) + ( 1 /} ( {K}_{2} \times {10}^{p H} )} & ( 8 )\end{matrix}$$

The proportion of each chlorine species can be plotted against pH. FIG. 1 of the accompanying drawings shows the familiar distribution of chlorine species as a function of pH. The key features of this distribution plot are:—
1) The maximum hypochlorous acid concentration is obtained at ˜pH5.5.
2) At pH 5.5 there is a small but significant level of hypochlorite ion.
3) At pH 5.5 there is a small but significant level of Cl₂ (aq).
4) Below pH 5.5 the Cl₂ (aq) level increases. Chlorine will gas off.

It was noted that the chloride ion concentration is present in the equations used to generate the distribution curves. By setting the chloride ion concentration as a variable it was possible observe it's affect on the distribution of chlorine species. FIG. 2 of the accompanying drawings illustrates the affect of increasing the chloride concentration by a factor of ten. The key features of this distribution plot are:—
1) The hypochlorite curve is changed very little.
2) The maximum hypochlorous acid concentration has shifted to pH 6.0.
3) The maximum level of hypochlorous acid has reduced.
4) The chlorine distribution curve has shifted by half of one pH unit to pH 5.5.
   This plot shows that in high chloride solutions (eg electrochemically generated hypochlorous acid) the hypochlorous acid level is limited and that chlorine is more readily generated.

FIGS. 3 and 4 illustrate the affect of lowering the chloride content. The key features of these distribution curves are:—
1) The hypochlorite distribution curve is changed lithe.
2) 100% hypochlorous acid is possible over a wider range of pH.
3) The chlorine distribution curve shifts one pH unit to lower pHs for each order of magnitude reduction in chloride concentration.

These plots show that stable hypochlorous acid can be more easily generated in low chloride solutions. The plot shows that less chlorine is present, even at low pH in low chloride solutions; hence the gassing off of chlorine will be greatly reduced. The gassing off of chlorine is a key route for the decomposition of hypochlorous acid solutions and limits how low the pH might be adjusted in the acid direction. This invention has shown that this route decomposition can be limited.
Hypochlorite can decompose via two routes:—
1) to chloride and chlorate
2) to oxygen and chloride
   Shifting the solution pH to lower pHs reduces the hypochlorite concentration in favour of hypochlorous acid. The hypochlorous acid can stabilised as described above. Since the hypochlorite concentration is much lower, the losses due to these two hypochlorite decomposition routes are reduced.

As with all chlorine solutions the decomposition processes are accelerated by light, high temperature and the presence of metallic ions. Thus the purity of raw materials appropriate packaging and storage are still important.

The improved stability of hypochlorous acid in low chloride solutions is only of benefit if we can lower the chloride content. When solutions of chlorine are prepared from chlorine gas, the chlorine dissolves in the water yielding hypochlorous acid and hydrochloric acid in equal stoichiometric quantities.

When sodium hypochlorite is manufactured, chlorine is dissolved in sodium hydroxide yielding sodium hypochlorite and sodium chloride in equal stoichiometric quantities. This means for a 14/15% solution of commercial sodium hypochlorite there is approximately 11% sodium chloride present. When organically stabilised chlorine is used as a source of chlorine, the chloride levels are low, but the organic backbone molecule can present the formulator with difficulties. Chlorinated phosphates also give low chloride solutions but they have a high phosphate to chlorine ratio which presents its own difficulties. They also have a high pH.

The present invention preferably utilises the high chlorine to chloride ratio in calcium hypochlorite. The calcium hypochlorite manufacturing process involves the reaction of chlorine with calcium hydroxide slurry. Calcium hypochlorite and calcium chloride are formed in equal stoichiometric quantities. The calcium chloride is more soluble than the calcium hypochlorite so predominantly resides in the brine liquor. The brine liquor was removed to reduce the chloride content. A second chlorination stage was done to enhance the hypochlorite concentration, involving the re-dispersion of the solids from the previous stage and reacting it with more chlorine. Calcium chloride was again removed when the solids were separated from the brine. This process resulted in a solid product containing 6-8% calcium chloride and 60-65% calcium hypochlorite.

When calcium hypochlorite was then dissolved in water an alkaline solution of calcium and hypochlorite ions was formed. The chlorine to chloride ratio is much higher than if the solution were prepared from, sodium hydroxide. Some of the calcium was precipitated as calcium hydroxide. This was separated off by filtration. The solution still contained a high level of calcium which was removed. The pH was adjusted to approximately 5-6 using phosphoric acid, which resulted in the precipitation of calcium phosphate. This was again removed by filtration.

The product was a near pure solution of hypochlorous acid. The pH may be adjusted to the desired value using further phosphoric acid or sodium hydroxide, as necessary. It is beneficial to have a low level of phosphate in the solution to act as a pH buffer. Stability tests have indicated that a low level of calcium stabilises any hypochlorite ions present.

The resulting stock solution was clear, the chlorine is present as free chlorine and it has only a weak halogen odour.

The stock solution can be used in the preparation of a wide range of products as detailed above in relation to the first and third aspects of the present invention. It is essential that dilutions are carried out with deionised water. Mains water and softened mains water contain chloride ions. Although only present at relatively low levels, low chloride levels are destabilising for low strength hypochlorous acid solutions.

When packaged and stored correctly, the product maintains a high free chlorine levels for greater than six months.

The stability of the composition according to the present invention was tested over a period of one month, the starting compositions containing 5000 ppm chlorine. The results are summarised in Table 1 below:

TABLE 1

| | | 8 Feb. 2011 | 9 Feb. 2011 | 11 Feb. 2011 | 14 Feb. 2011 | 18 Feb. 2011 | 24 Feb. 2011 | 01 Mar. 2011 | 07 Mar. 2011 |
|---|---|---|---|---|---|---|---|---|---|
| | Day | 1 | 2 | 4 | 7 | 11 | 17 | 22 | 28 |
| DPD1 | ppm Cl2 | 5000 | 5000 | 5000 | 4600 | 4500 | 4500 | 4500 | 4000 |

The results show an 80% retention in HOCl, far higher than the non-stable electrolysis-prepared aqueous solutions of the prior art.

### EXAMPLE 2

### Preparation of Stable HOCl Solution from Anhydrous Composition of 75% Assay Pure Calcium Hypochlorite and Citric Acid

“x” grams of a 75% assay pure calcium hypochlorite, such as that sold by Arch Chemicals, was mixed with “y” grams of citric acid and then placed in a water soluble, non-halogen demanding coating, such as gelatin or a gelatin substitute. Upon contact with water of a given volume “z” and a pH of “A”, HOCl is produced at a specific ppm and pH, as shown in Table 2 below. A pH of 5.5 was achieved using de-ionised water.

TABLE 2

| | | g/l | | | Weight ratio |
|---|---|---|---|---|---|
| | g/L of cal | of citric | | ppm after 1 | of CAmh:cal |
| ppm¹ | hypo ULC | acid² | Initial ppm³ | hour³ | hypo |
| 250 | 0.44 | 0.37 | 275 | 263 | 0.84 |
| 1000 | 1.76 | 1.45 | 1000 | 870 | 0.82 |
| 2000 | 3.52 | 2.9 | 2000 | 1025 | 0.82 |
| 4000 | 7.04 | 5.7 | 3600 | 1750 | 0.81 |

¹The cal hypo is added based upon that which is required to make the stable HOCl solution to give these ppms.
²This is citric acid monohydrate (Camh).
³ppm of chlorine is measured via DPD (diethyl-p-phenylene diamine tablets).

1. A composition comprising a stable solution of hypochlorous acid wherein the ratio of chloride to hypochlorous acid is at a maximum of 1 chloride:3 hypochlorous acid and the pH of the solution is maintained at a pH of between 3.5 to 7.0 whereby the stable HOCl retains its antimicrobial activity for a period of at least one month.

2. (canceled)

3. A composition as claimed in claim 1 wherein the chloride to hypochlorous acid ratio has a chloride level of 1:8 chloride to hypochlorous acid.

4. A composition as claimed in claim 1 wherein the composition further comprises one or more additional components selected from the group consisting of a halogen stable anionic or cationic surfactant, moisturizing agent, fragrance, emollient, chelating agent, colourant, optical enhancer, biocide or adjuvant, chlorite donor, halogen releaser and an equilibrium stabilizer.

5. (canceled)

6. A process for preparing a stable aqueous solution of hypochlorous acid, the process comprising the steps of adding a source of chlorine to water, manipulating the chloride levels in the resulting solution to be at a maximum of 1 chloride:3 hypochlorous acid, and controlling the pH of the solution to between 3.5 to 7.0.

7. A process according to claim 6 wherein the chlorine source is calcium hypochlorite, water is deionized water and phosphoric acid maintains the pH of the solution.

8. (canceled)

9. (canceled)

10. A process for preparing an aqueous solution of hypochlorous acid having low chloride levels, the process comprising the steps of adding solid hypochlorite and at least one phosphate buffer to deionised water.

11. (canceled)

12. (canceled)

13. A process according to claim 6, further comprising the steps:
adding chlorine to calcium hydroxide to provide a solution of calcium hypochlorite, calcium chloride and water;
removing the calcium chloride in solution to provide solid calcium hypochlorite; dissolving the calcium hypochlorite in water to provide an alkaline solution of calcium and hypochlorite ions;
precipitating calcium as calcium hydroxide to enable its subsequent removal;
adjusting the pH of the resultant solution of hypochlorite ions to around 5-6 to precipitate any remaining calcium to enable its subsequent removal; and
adjusting the pH of the solution of hypochlorous acid to a pH of between 3.5 and 7.

14. (canceled)

15. A solid composition for use in the preparation of a stable solution of hypochlorous acid, the composition comprising a solid halogen source and a separate solid acid, the halogen source and acid being encapsulated, together or separately, by a non-halogen demanding coating.

16. (canceled)

17. A composition as claimed in claim 15 wherein the acid is selected from the group consisting of citric, adipic, oxalic, malonic, succinic, glutaric, pimelic, suberic and phosphoric acid.

18. (canceled)

19. (canceled)

20. (canceled)

21. A composition as claimed in claim 15 wherein the coating is water-soluble.

22. A composition as claimed in claim 15 wherein the halogen source and acid are encapsulated within exines obtained from spores or pollens.

23. The use of a stable aqueous solution of hypochlorous acid as claimed in claim 1 as an antimicrobial agent.

24. The use of a stable aqueous solution of hypochlorous acid as claimed in claim 1 as a cleaning or sterilising agent.

25. A non-woven material impregnated with a composition as claimed in claim 1.

26. A method of sterilizing an animate or inanimate object comprising contacting the object with a composition as claimed in claim 1.

27. The use of a stable aqueous solution of hypochlorous acid as produced from the composition of claim 15 as an antimicrobial agent.

28. The use of a stable aqueous solution of hypochlorous acid as produced from the composition of claim 15 as a cleaning or sterilising agent.

29. A non-woven material impregnated with a composition as claimed in claim 15.

30. A method of sterilizing an animate or inanimate object comprising contacting the object with a composition as claimed in claim 15.
